(19) United States
(12) Patent Application Publication
(10) Pub. No.: US 2006/0222704 A1
(43) Pub. Date: Oct. 5, 2006

(54) System and a method for labeling a substrate

(76) Inventors: Marcos A. Barreto, Aquadilla, PR (US); Lilliam E. Gonzalez, Isabela, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/223,267

(22) Filed: Sep. 9, 2005

Related U.S. Application Data
(63) Continuation-in-part of application No. 11/097,087, filed on Mar. 31, 2005.

(51) Int. Cl. A61K 9/28; A61K 9/20; B05D 3/02

(52) U.S. Cl. 424/464; 427/2.14

(57) ABSTRACT

A method for labeling a substrate includes positioning the substrate adjacent to an inkjet material dispenser, and selectively jetting an edible ink onto the substrate with the inkjet material dispenser. The edible ink is configured to exhibit a known fluorescent emissive profile when exposed to a visible light.

### RELATED APPLICATIONS

The present application is a continuation-in-part of application Ser. No. 11/097,087, filed on Mar. 31, 2005, which application is incorporated by reference herein in its entirety.

### BACKGROUND

Pharmaceutical products such as pills and capsules traditionally include a number of markings. Marks or printed information on pharmaceutical products typically include information such as logos, names, or bar codes that may be used to identify the type, dosage, and/or source of the pharmaceuticals. These markings also aid in the dispensing and administration of pharmaceuticals to patients.

In order to mark or otherwise label pharmaceutical products such as pills and capsules, the methodology has to be approved by the food and drug administration (FDA). The FDA has a number of lists containing approved colorants and labels that may be used to mark or otherwise label pharmaceutical products.

Traditional methods of marking pharmaceutical products such as pills and capsules include coloring the pharmaceutical products with FDA certified colorants, altering the surface appearance of the pharmaceutical products through engravings, applying a label to the surface of the pharmaceutical products, or painting the pharmaceutical product.

While traditional methods are somewhat effective in marking or otherwise distinguishing pharmaceutical products, traditional methods of marking pharmaceutical products significantly compromise the outward appearance of the pharmaceutical products. Additionally, traditional methods do little to facilitate the control of counterfeit production and fraudulent dispensing of pharmaceuticals, since these outer markings are easily identifiable and reproducible. Moreover, a majority of the traditional methods and formulations for marking or otherwise labeling pharmaceutical products necessitate contact with the pharmaceutical. Any such contact with the pharmaceutical products increases the likelihood of causing physical or chemical damage to the pharmaceutical product.

### SUMMARY

In one of many possible embodiments, a method for labeling a substrate includes positioning the substrate adjacent to a material dispenser, and selectively dispensing an edible ink onto the substrate with the material dispenser. The edible ink is configured to exhibit a known fluorescent emissive profile when exposed to a known range of visible light.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a perspective view of a pharmaceutical product labeled according to teachings of the prior art.

FIGS. 2A and 2B are emission profiles of fluorescent dyes irradiated with ultraviolet light, according to one exemplary embodiment.

FIG. 3A is an emission profile that occurs when both of the fluorescent dyes of FIGS. 2A and 2B are irradiated simultaneously, according to one exemplary embodiment.

FIG. 3B illustrates a number of superimposed emission profiles showing modifications in the emission profiles due to the number of dye drops present, according to one exemplary embodiment.

FIG. 4 is a flow chart illustrating an exemplary method for forming an edible ink for marking a product, according to one exemplary embodiment.

FIG. 5 is a flow chart illustrating an exemplary method for marking a product with a spectral fingerprint using fluorescent compounds, according to one exemplary embodiment.

FIG. 6 is a perspective view illustrating a pharmaceutical marking system, according to one exemplary embodiment.

FIG. 7 is an emission profile illustrating composite images containing a plurality of fluorescent colorants, according to one exemplary embodiment.

FIG. 8 is a flow chart illustrating a method for authenticating a pharmaceutical product, according to one exemplary embodiment.

FIG. 9 is a number of emission profiles illustrating a method for varying a measured emissive profile based on an illumination area of a UV light, according to one exemplary embodiment.

FIG. 10 is an emission profile of a fluorescent dye irradiated with a visible green light and an ultraviolet light, according to one exemplary embodiment.

Throughout the drawings, identical reference numbers designate similar, but not necessarily identical, elements.

### DETAILED DESCRIPTION

An exemplary system and method for forming a spectral fingerprint on an object to be tracked and/or verified is disclosed herein. More specifically, an ink is disclosed that is not visible to the naked eye under normal white light conditions; however, when irradiated with a known wavelength of light, the ink fluoresces and provides an identifiable spectral profile. According to one exemplary embodiment, the ink is non-toxic, edible, and made of compounds listed in the Generally Regarded as Safe (GRAS) list sponsored by the Food and Drug Administration (FDA) and configured to be applied to pharmaceutical products

As used in the present specification and the appended claim, the term “edible” ink is meant to be understood broadly as any composition that is suitable for human consumption and is non-toxic. Similarly, the phrase “edible ink” is meant to be understood as any ink that is suitable for human consumption and complies with applicable standards such as food, drug, and cosmetic (FD&C) regulations in the United States and/or Eurocontrol experimental centre (E.E.C.) standards in the European Union. Additionally, the term “invisible” is meant to be understood broadly as meaning any image, color, or shading that, when viewed by the naked eye, is not prominent or readily noticeable. The term “jettable” is meant to be understood both in the present specification and in the appended claims as any material that may be selectively deposited by any digitally addressable inkjet material dispenser.

In the following description, for purposes of explanation, numerous specific details are set forth in order to provide a thorough understanding of the present system and method for optically tracking objects using a spectral fingerprint of fluorescent compounds. It will be apparent, however, to one skilled in the art, that the present method may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

Exemplary Structure

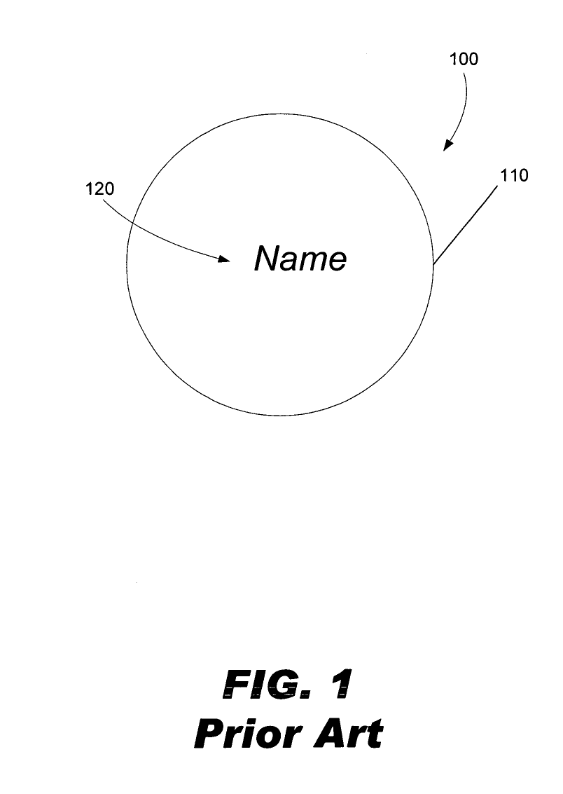

FIG. 1 illustrates a traditionally marked pharmaceutical product (100). As shown in FIG. 1, the pharmaceutical product (100) includes a pill (110) having a product name (120) printed thereon. As shown in FIG. 1, the product name (120) or other surface markings compromise the outward appearance of the pharmaceutical product (100).

Traditionally, the incorporation of a product name, a product coating color, and/or or product design has been used to discourage the production of counterfeit pharmaceuticals. However, the threat of counterfeit attacks, regulation, and possible liability are driving a desire for enhanced pharmaceutical authentication measures. According to one exemplary embodiment of the present system and method, a spectral fingerprint is formed with a number of fluorescent compounds to track and authenticate pharmaceuticals and other similar products.

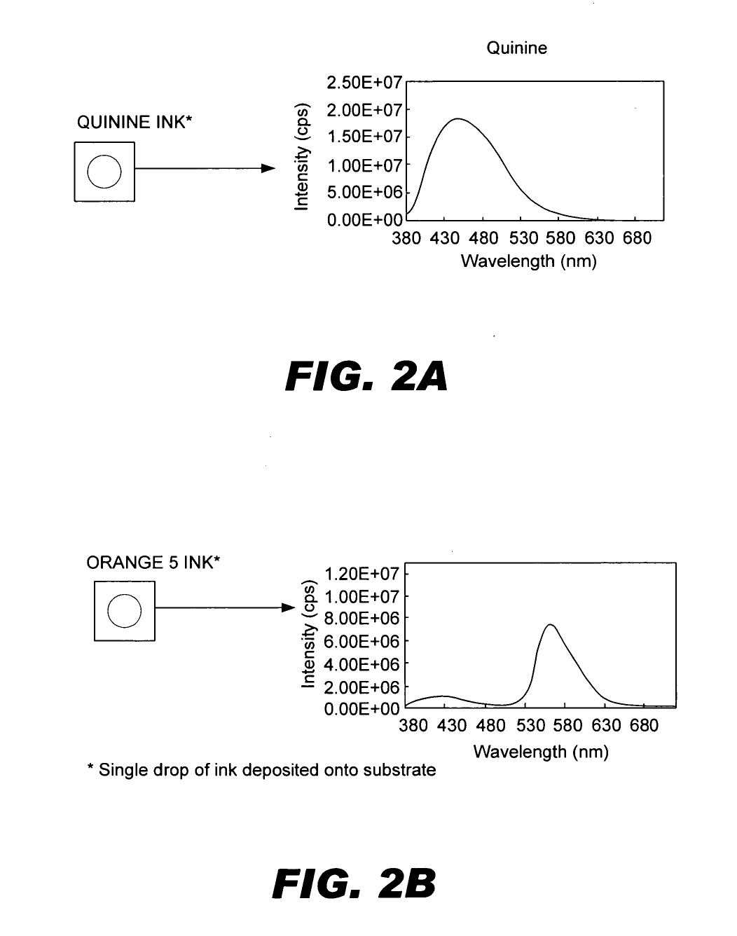

As illustrated in FIGS. 2A and 2B, emission profiles for single drops of a number of fluorescent dyes is shown. As shown in FIG. 2A, a fluorescent dye in the form of Quinine ink was deposited on a substrate and irradiated with UV light operating at a wavelength of 365 nm. The fluorescent emission profile of the substrate was then examined by a spectrofluorometer. According to one exemplary embodiment, the spectrofluorometer (not shown) includes an excitation light source configured to operate at a known wavelength and a detector configured to measure the fluorescence of a desired object. According to one exemplary embodiment, the detector includes, but is in no way limited to, a charge coupled device (CCD) detector. According to the exemplary embodiment illustrated in FIG. 2A, the quinine ink was irradiated with UV light having a wavelength of approximately 365 nm generated by a spectrofluorometer. Once the ink was irradiated, the fluorescent emission profile was sensed and graphically generated.

As shown in FIG. 2A, the quinine ink generated a fluorescent emission profile that peaks between 430 and 480 nm exhibiting an intensity of approximately 2.00 E+07 cycles per second (cps). After peaking, the fluorescent emission profile slowly dissipates in intensity as the wavelength increases. As illustrated in FIG. 2A, the fluorescent emission profile is reduced to approximately 5.00 E+06 cps at a wavelength of 530 nm and approaches 0.00 E+00 at a wavelength of approximately 630 nm.

A similar test was performed on a single drop of orange 5 ink, as illustrated in FIG. 2B. As illustrated in FIG. 2B, the sample of orange 5 ink, irradiated with UV light having a wavelength of approximately 365 nm and examined by a spectrofluorometer produced a fluorescent emission profile that is substantially different than that of the quinine ink illustrated in FIG. 2A. As shown in FIG. 2B, the orange 5 ink produces substantially negligent intensity at wavelengths between approximately 380 nm and 510 nm. However, beginning with wavelengths of approximately 530 nm, the intensity of the orange 5 ink being irradiated with UV light having a wavelength of approximately 365 nm increases dramatically and peaks at an intensity of approximately 8.00 E+06 with wavelengths of approximately 565 nm. The fluorescent emission profile than decays somewhat rapidly in intensity to approximately 0.00 E+00 with wavelengths of approximately 650 nm and higher.

Comparing the fluorescent emission profiles of the quinine ink illustrated in FIG. 2A and the orange 5 ink illustrated in FIG. 2B shows that various inks that are susceptible to being excited when exposed to UV illumination produce very different fluorescent emission profiles. These differences in fluorescent emission profiles may be used to generate identifiable spectral fingerprints that may be formed on pharmaceutical or other products and may be used to track and authenticate the products, as will be explained in further detail below.

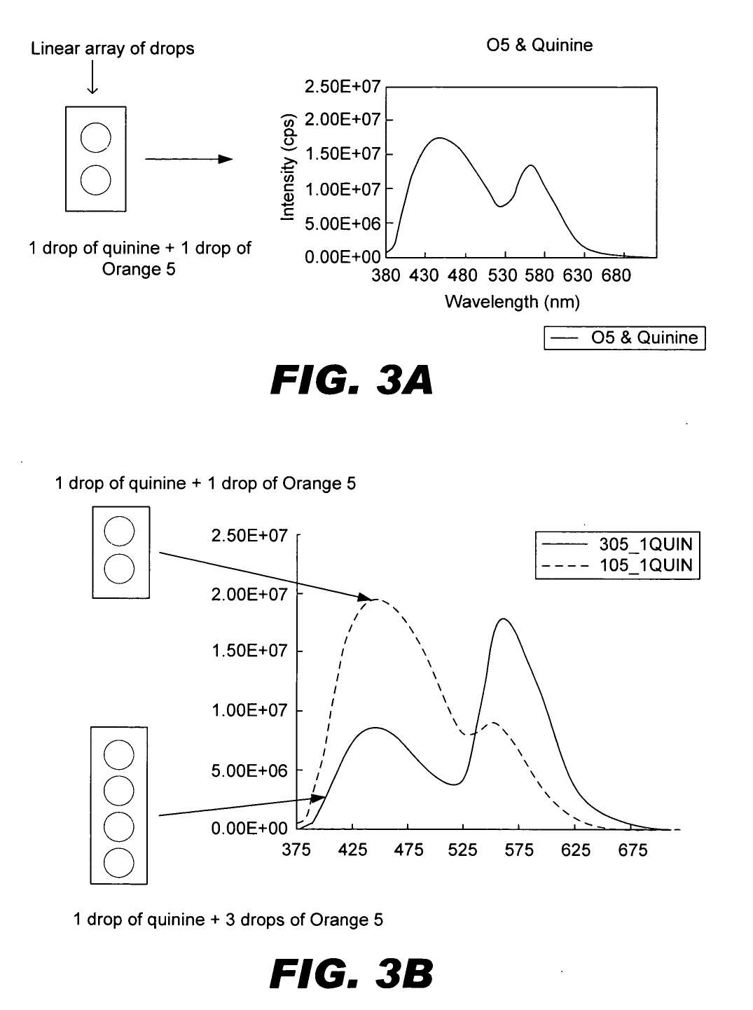

As shown in FIG. 3A, the various inks exhibiting different fluorescent emission profiles may be combined to produce a single emission profile. As illustrated in FIG. 3A, one drop of quinine and one drop of orange 5 were formed in a linear array of drops and then irradiated with UV light having a wavelength of approximately 365 nm and examined by a spectrofluorometer. The resulting fluorescent emission profile is illustrated in FIG. 3A. As shown, the linear array produces a fluorescent emission profile having a plurality of peaks and valleys that correspond to the peaks and valleys of the independent ink drops. More specifically, as illustrated in FIG. 3A, the linear array including one single drop of quinine and one single drop of orange 5 produces a fluorescent emission profile having a first peak between 430 and 480 nm exhibiting an intensity of approximately 2.00 E+07 cycles per second (cps) and a second peak at wavelengths of approximately 565 nm having an intensity of approximately 8.00 E+06 cps.

Selectively varying the number and type of ink drops in the linear array of drops will modify the resulting fluorescent emission profile, as is illustrated in FIG. 3B. As shown in FIG. 3B, a fluorescent emission profile of a linear array containing one drop of quinine and one drop of orange 5, similar to that illustrated in FIG. 3A, was compared to a fluorescent emission profile of a linear array containing one drop of quinine and three drops of orange 5. As shown in FIG. 3B, the concentrations of the various inks have a large impact on the resulting fluorescent emission profile. Specifically, the inclusion of 3 drops of orange 5 in the linear array modified the intensity of the fluorescent emission profile. Adding three drops of orange 5 greatly increased the intensity of the emission profile between the wavelength ranges of approximately 525 nm and 625 nm, the area corresponding with the orange 5. As shown, the intensity level of the peak present between approximately 525 nm and 625 nm, typically associated with the influence of the orange 5, increased from approximately 8.00 E+06 to an intensity value of approximately 1.80 E+07. Additionally, the intensity value of the emission profile between approximately 400 nm and 500 nm, typically attributed to the influence of the quinine, experienced a reduction in intensity from approximately 2.00 E+07 to approximately 8.00 E+06. These unique variations in fluorescent emission profile based on the concentrations of a plurality of fluorescing materials may be used to authenticate pharmaceutical products due to its difficulty to duplicate. In addition, data can be encoded within the fluorescing patterns to enable product tracking features. Exemplary compositions of the fluorescing inks will be described below, followed by an explanation of an exemplary method for marking and tracking pharmaceuticals and other products with the present system and method.

Exemplary Composition

According to one exemplary embodiment, the above-mentioned UV fluorescing inks are to be applied to pharmaceuticals. Consequently, according to this exemplary embodiment, the components of the UV fluorescing ink are edible, making the fluorescing inks edible inks, as defined herein. According to one exemplary embodiment, the present edible ink includes at least two main components: an ink vehicle and a colorant.

The ink vehicle component of the present edible ink provides a liquid solution that facilitates dispersion and dissolution of the colorant while enabling the selectively controlled transport of the colorant from a material dispenser to a pharmaceutical product or other printing substrate. Additionally, the ink vehicle can also act as a binder to affix the colorant to the pharmaceutical product or other print substrate. The components of the ink vehicle may be made of any edible compound with substantially non-visible properties when applied to a pharmaceutical product or other printing substrate. Examples of these compounds are listed in the Generally Regarded as Safe (GRAS) list sponsored by the food and drug administration (FDA). According to one exemplary embodiment, the ink vehicle includes at least a solvent, but may also include additives configured to enhance various properties and characteristics of the resulting ink. Property enhancing additives that may form a part of the ink vehicle may include, but are in no way limited to, surfactants, buffers, and/or humectants.

The solvent component of the ink vehicle is included in the present edible ink for dispersion and transport of the colorant as well as any additives. The vehicle solvent, according to one exemplary embodiment, imparts a jettable viscosity to the edible ink while also evaporating at a rate sufficient to make a printed image resistant to smudging soon after it is deposited on a pharmaceutical product or other ink receiving substrate. According to one exemplary embodiment, the solvent comprises water, thus creating a water-based vehicle. In addition to low cost, water is effective as a solvent for many additives, greatly reduces inkjet dispenser compatibility issues, effectively suspends colorants, and effectively controls drying rates of the ink. More specifically, a water-based vehicle may comprise from 20% by volume water up to about 90% by volume water. In another exemplary embodiment, the solvent component of the ink vehicle includes a mixture of water and an alcohol, such as ethyl alcohol. The addition of an alcohol to a solvent affects the viscosity and drying rate of the ink vehicle, as well as acting as a surfactant.

Surfactants and emulsifiers may be added to the solvent component of the present edible ink in order to facilitate dispersion and/or dissolution of the colorant and any other additive in the solvent. Typically, an edible alcohol may be used as the vehicle surfactant including, but in no way limited to, ethyl alcohol, glycerol, methyl alcohol, isopropyl alcohol, and butyl alcohol. Ethyl alcohol, for example, decreases the surface tension of water, thereby allowing a colorant and other additives to dissolve and/or disperse throughout the water more easily. The ethyl alcohol may also facilitate the jettability of the present edible ink, according to one exemplary embodiment. Moreover, other edible compounds besides alcohols may also be used as the surfactant or emulsifier, including, but in no way limited to, lecithin, crillet, polyoxyethylene sorbitan monostearate (TWEEN), xanthan gum, sorbitol, and starches such as maize starch, corn starch, and potato starch. According to one exemplary embodiment, a surfactant or emulsifier may be present in a concentration of up to about 20% by volume of the ink vehicle. In one particular embodiment, the surfactant or emulsifier comprises ethyl alcohol in a concentration of about 17% to about 20% by weight. In another aspect, ethyl alcohol comprises from about 13% to about 17% by weight of the vehicle.

In addition to solvents, surfactants, and emulsifiers, the ink vehicle may also include a pH buffer to control the pH level of the edible ink. The pH level of the edible ink may be adjusted to vary, among other things, the fluorescence intensity of the colorant. According to one exemplary embodiment, an acid is used as a buffer to increase the acidity of the ink. Increasing the acidity level of the ink intensifies the fluorescence of some colorants, such as quinine sulfate, making the edible ink fluoresce brighter thereby making it more visible under UV light. The concentration of acid used may vary depending on the desired fluorescence intensity but typically comprises up to about 1% by weight of the vehicle. In one specific embodiment, the buffer comprises sulfuric acid in a concentration of about 0.4% by weight of the vehicle. In another embodiment, the acid is present in the vehicle in a concentration of about 5 milligrams per milliliter of water.

A humectant may also be included in the present ink vehicle to control the moisture content and viscosity thereof, according to one exemplary embodiment. The ink vehicle typically dries by evaporation once it is disposed on the pharmaceutical product or other substrate surface; however, drying prior to printing decreases viscosity and thus may inhibit the jettability of the edible ink. Therefore, a humectant may be included in the vehicle to keep the edible ink from premature drying. The humectant may include, but is in no way limited to glycerin, sorbitol, mannitol, or any other edible humectant. According to one exemplary embodiment, the humectant can be present in the vehicle in a concentration of up to approximately 5% of the vehicle by volume. According to another exemplary embodiment, the humectant includes glycerin in a concentration of approximately 3% by volume, or about 4% by weight, of the ink vehicle.

According to one exemplary embodiment, the vehicle component of the present edible ink may also include other additives as needed including, but in no way limited to, driers, thinners, waxes, lubricants, reducing oils and solvents, body gum and binding varnish, antioxidants and anti-skinning agents, resins, and/or binders.

The present edible ink also includes an edible colorant component configured to produce a desired emission profile when exposed to UV light. According to one exemplary embodiment, the colorant component is not visible to the human eye when applied to the pharmaceutical product or other substrate, either because it is colorless or because it is the same color as the pharmaceutical product or printing substrate. Suitable colorants include any edible compounds, or combinations thereof, that naturally fluoresce when exposed to UV light including, but in no way limited to, riboflavin, riboflavin phosphate including riboflavin 5′-phosphate, pyridoxine hydrochloride, folic acid, quinine sulfate, niacin, nicotinamide, D&C Orange No. 5, or any appropriate combination thereof. The afore-mentioned fluorescent colorants are also water-soluble, further facilitating their incorporation into a water-based ink vehicle.

While the above-mentioned exemplary compositions are configured for application to a pharmaceutical product, the above-mentioned edible ink may be used to mark, track, and authenticate any number products that may come in contact with a consumer's mouth including, but in no way limited to, food products, pharmaceutical coverings, or dental products. Additionally, the present system and method may be incorporated with objects not restricted by the food, drug, and cosmetic (FD&C) regulations in the United States and/or Eurocontrol experimental centre (E.E.C.) standards in the European Union. When applied to non-restricted components, the present marking ink may include any number of colorants that fluoresce when exposed to UV light.

The component concentrations mentioned above are merely given as examples and are in no way meant to limit the contemplated concentrations. Rather, the concentration of the colorant or other components can be lower if less intense fluorescence is desired or higher if more intense fluorescence is desired.

Exemplary Composition Forming Methods

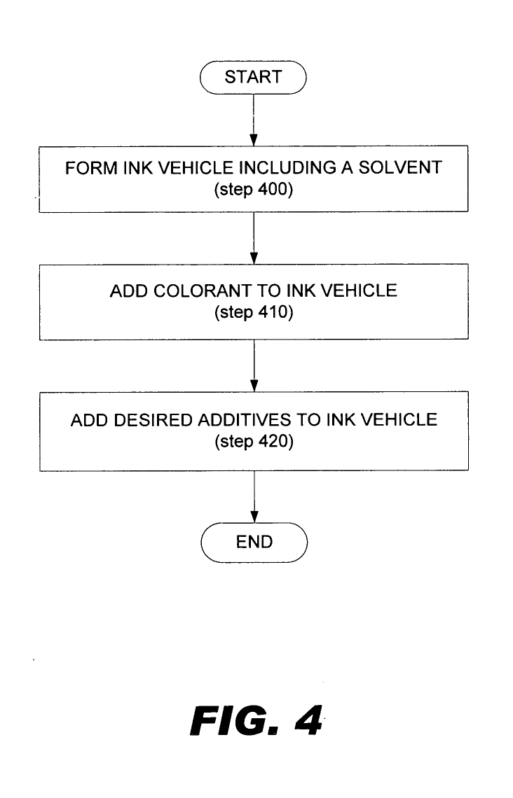

FIG. 4 illustrates a method for forming the present edible ink that may be used for tracking and authentication, according to one exemplary embodiment. The edible ink can be formed in a simple process of combining a solvent, colorant, and additives. As shown in FIG. 4, the present edible ink may be formed by first, forming an ink vehicle including a solvent (step 400). Once the ink vehicle is formed, a colorant may be added (step 410) followed by a number of desired additives (step 420). While FIG. 4 illustrates the additives being included in the present edible ink after the colorant is added (step 410), additives, such as a buffer, humectant, or surfactant can be added to the mixture before the colorant is added, after the colorant is added, or both.

According to one exemplary embodiment, the present edible ink can be made by performing the following steps: First, an aqueous acid or buffer solution is prepared. Colorant is then added to the acid solution and mixed. Next, a surfactant is added to the solution. Finally, the solution is mixed until the colorant is well dissolved. In another exemplary embodiment, the ink can be formed by performing the following steps: First, the ink vehicle is prepared by combining and mixing a solvent, surfactant, and humectant. The colorant is then added and mixed until dissolved.

Exemplary Implementation and Operation

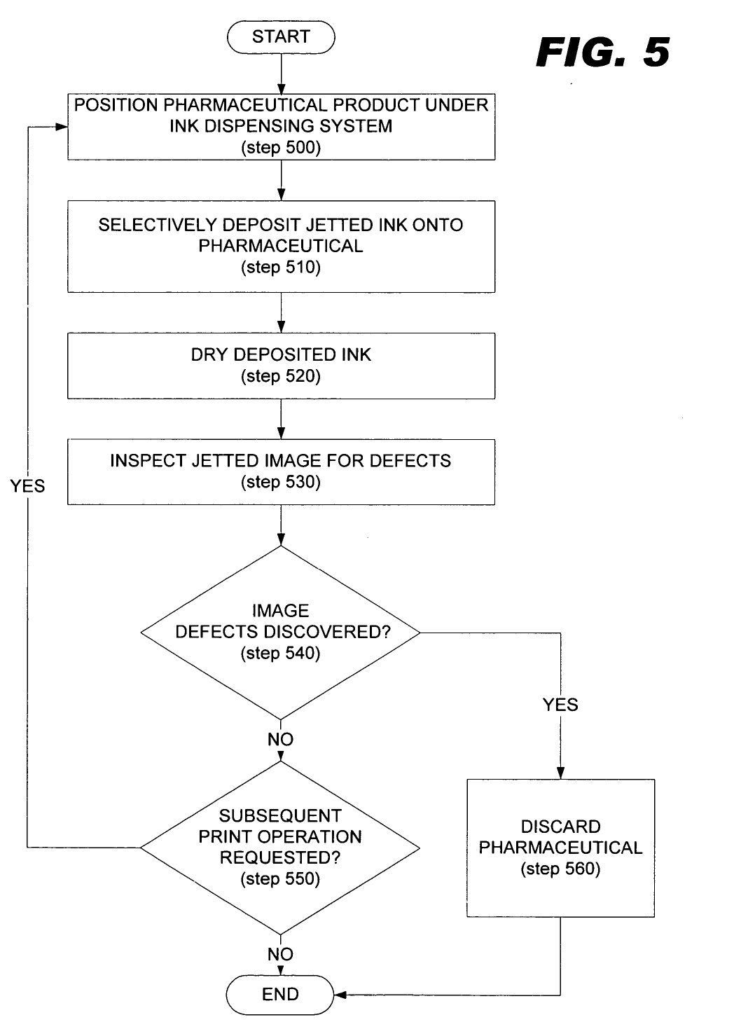

Once formed, the present edible ink may be dispensed onto a pharmaceutical product or other substrate to form a desired image that is invisible to the naked eye under normal white light conditions but visibly fluoresces and exhibits a distinct fluorescent emission profile when exposed to a known wavelength of light. FIG. 5 illustrates a method for implementing the above-mentioned tracking and authentication techniques to mark a pharmaceutical product, according to one exemplary embodiment. As shown in FIG. 5, the present method begins by positioning a pharmaceutical product under an ink dispensing system (step 500). Once positioned, the ink dispensing system selectively deposits the edible ink onto the pharmaceutical product (step 510) where it subsequently dries (step 520). Upon deposition of the edible ink, the image is inspected for defects (step 530) and a determination is made as to whether the printing system has satisfactorily completed its ink dispensing operation by determining whether any image defects were discovered (step 540). If image defects are discovered (YES, step 540), the printing system discards the defective pharmaceutical (step 560). If, however, no defect is detected on the pharmaceutical (NO, step 540), the system then determines if a subsequent print operation is requested (step 550). If another print operation is requested, the present method returns again to step 500 and the pharmaceutical is again positioned under an ink dispensing system to receive an ink. If, however, no subsequent print operation is requested (NO, step 550), the printing of the pharmaceutical is complete. The above-mentioned steps will now be described in further detail below.

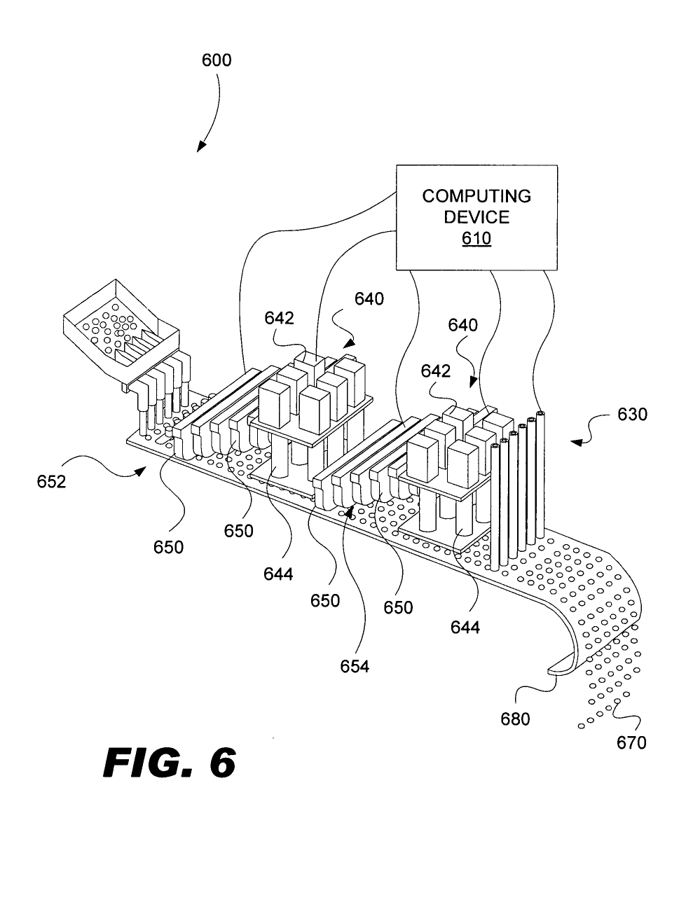

As shown in FIG. 5, the present exemplary method for printing an edible ink on a pharmaceutical product begins by positioning the pharmaceutical product to receive the edible ink under the ink dispensing system (step 500). FIG. 6 illustrates an exemplary ink dispensing system (600), according to one exemplary embodiment. As illustrated in FIG. 6, the exemplary ink dispensing system (600) includes a number of ink dispensers (650) containing one of the above-mentioned edible inks (652, 654) disposed therein. As shown in FIG. 6, a pharmaceutical product (670) may be positioned under the ink dispensers (650) by a moveable substrate (680). Alternatively, an operator may manually place the pharmaceutical product (670) adjacent to the ink dispensing system (600). While the present embodiment is described in the context of marking a pharmaceutical product (670) with an edible ink (652, 654), the present system and method may be used to mark any number of items with the present edible ink (660) including, but in no way limited to, food products, dental products, etc.

Once the pharmaceutical product is positioned under the desired ink dispenser (650), the ink dispenser is directed to selectively deposit the above-mentioned ink onto the pharmaceutical (step 510; FIG. 5). As shown in FIG. 6, the exemplary ink dispensing system (600) includes a computing device (610) controllably coupled to the components of the system. The computing device (610) that is controllably coupled to the components of the ink dispensing system (600) controls the selective deposition of the edible ink (652, 654) on the pharmaceutical product (step 510; FIG. 5). A representation of a desired image or label may be formed using a program hosted by the computing device (610). That representation may then be converted into servo instructions that are then housed in a processor readable medium (not shown). When accessed by the computing device (610), the instructions housed in the processor readable medium may be used to control a number of servo mechanisms (not shown) as well as the movable substrate (680) and the ink dispensers (650). The computing device (610) illustrated in FIG. 6 may be, but is in no way limited to, a workstation, a personal computer, a laptop, a personal digital assistant (PDA), or any other processor containing device.

As an image is printed on a pharmaceutical product, the computing device (610) may controllably position the moveable substrate (680) and direct one or more of the ink dispensers (650) to selectively dispense an edible ink at predetermined locations on the pharmaceutical product (670) as digitally addressed drops, thereby forming the desired image. The ink material dispensers (650) used by the present printing system (600) may be any type of ink dispenser configured to perform the present method including, but in no way limited to, thermally actuated inkjet dispensers, mechanically actuated inkjet dispensers, electrostatically actuated inkjet dispensers, magnetically actuated dispensers, piezoelectrically actuated dispensers, continuous inkjet dispensers, etc. Additionally, the ink-jet material dispenser can be heated to assist in dispensing the edible ink. Moreover, the present edible ink can be distributed using any number of printing processes including, but in no way limited to, inkjet printing, lithography, screen printing, gravure, flexo printing, or stamping.

Once the desired image or pattern is formed on the pharmaceutical product (670), the ink dries (step 520; FIG. 5) and the pharmaceutical product is inspected for image defects (step 530; FIG. 5). As illustrated in FIG. 6, the exemplary printing system (600) includes an ink vision system (640) associated with each inkjet dispenser (650). According to one exemplary embodiment, each ink vision system (640) includes the components of a spectrofluorometer including, but not limited to, an excitation light source (not shown) configured to operate at a known wavelength and a CCD detector (642) configured to measure the fluorescence of a desired object. Additionally, the ink vision system (640) may include optics (644) associated with the CCD detector (642). According to one exemplary embodiment, the image defect inspection method (step 530; FIG. 5) includes the vision system (640) analyzing the fluorescent emission profile of each pharmaceutical product printed to determine if the emission profile matches an expected profile for the requested ink deposition. Analyzing the fluorescent emission profile of a printed image may include, but is not limited to, decomposing the image between multiple wavelengths and comparing the wavelength intensities to a number of expected intensities set for the amount and combination of ink that has been printed on the pharmaceutical product (670).

After the jetted image is inspected (step 530; FIG. 5), the present exemplary printing system (600) determines if any image defects were sensed during the inspection (step 540; FIG. 5). As mentioned previously, image defects, according to one exemplary embodiment, correspond to a resulting emission profile that does not substantially match an expected emission profile. According to one exemplary embodiment, the emission profile detected by the vision system is compared to an expected emission profile stored in the computing device (610). If an image defect is detected by the exemplary printing system (YES, step 540; FIG. 5), the pharmaceutical is discarded (step 560; FIG. 5). According to one exemplary embodiment, a vacuum reject system (630) is disposed adjacent to the moveable substrate (680) and is configured to selectively remove defective pharmaceutical products (670) by vacuuming the pharmaceutical product through a vacuum tube.

Once the initial inspection process (step 540; FIG. 5) has been performed, the present exemplary printing system (600) determines if additional print operations have been selected. If an ink design incorporating a plurality of ink depositions is desired (YES, step 550; FIG. 5), the pharmaceutical product (670) is again positioned under an ink dispensing system (step 500; FIG. 5) in preparation for receiving a jettable ink. As illustrated in FIG. 6, the exemplary printing system (600) may include a number of inkjet dispenser (650) stations and a plurality of vision systems (640). According to one exemplary embodiment, a plurality of inkjet dispensers (650) having various inks exhibiting different fluorescent emission profiles. According to one exemplary embodiment, a first group of inkjet dispensers (650) include a first quinine based ink (652), and a second group of inkjet dispensers (650) include a second orange 5 based ink (654). According to this exemplary embodiment, various drops of ink may be selectively deposited on the surface of a pharmaceutical product (670) to generate a desired fluorescent emission profile. That selective fluorescent emission profile may then be used to track and/or authenticate the genuineness of the selected pharmaceutical product (670). When it is determined that no further ink deposition is requested, the process, with respect to the identified pharmaceutical product (670) is complete. Any number of subsequent packaging and/or processing operations may then be performed on the pharmaceutical product (670). While the present exemplary method is described as introducing the fluorescent compounds or inks to the pharmaceutical product individually, the fluorescent compounds can be applied either individually or in combination to produce an “invisible” pattern that can be authenticated under UV light. Further, the pharmaceutical product can be authenticated in a non-destructive test at any point during its supply chain by the use of a spectrofluorometer.

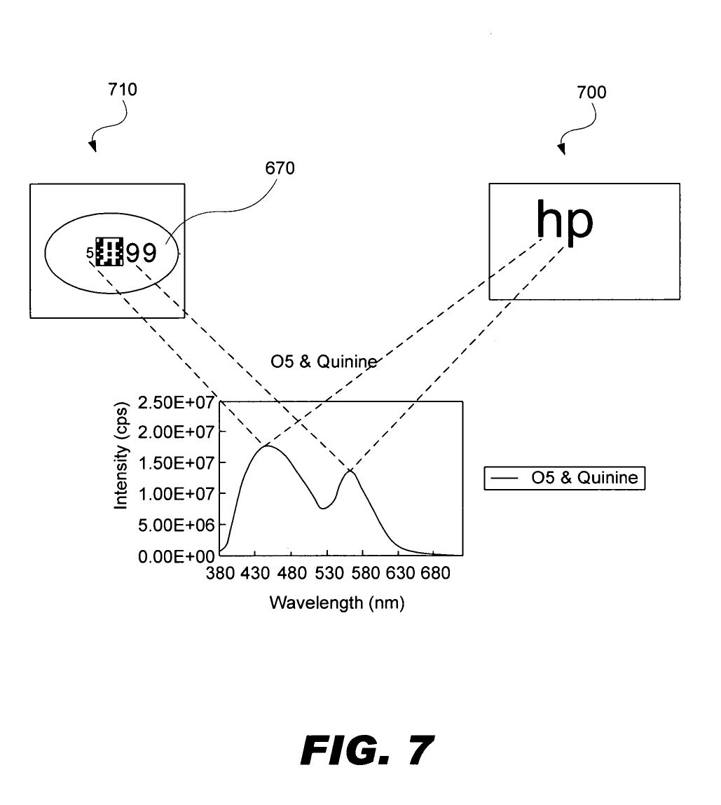

FIG. 7 illustrates a number of “images” that may be produced with the present exemplary system and method. According to one exemplary embodiment, illustrated in FIG. 7, the present system may form a name or a mark (700) on a pharmaceutical product (670) or other substrate to be tracked and/or authenticated. Either all or merely a portion of the pharmaceutical product (670) may be printed of one or more UV fluorescing inks to produce a unique spectral profile when excited with illumination of a known wavelength. Alternatively, the image formed on the pharmaceutical product (670) or other substrate to be tracked and/or authenticated may be a data carrying image such as a barcode or a data matrix (710). As illustrated in FIG. 7, a data matrix (710) is formed on the pharmaceutical product (670). A data matrix (710) is meant to be understood as a two-dimensional barcode which can store from 1 to about 2,000 characters. The data matrix (710) may then be scanned by a spectrofluorometer for a spectral fingerprint, and scanned by a 2-D UV scanner to interpret the stored information. This way, a double counterfeit protection may be created for each pharmaceutical product (670).

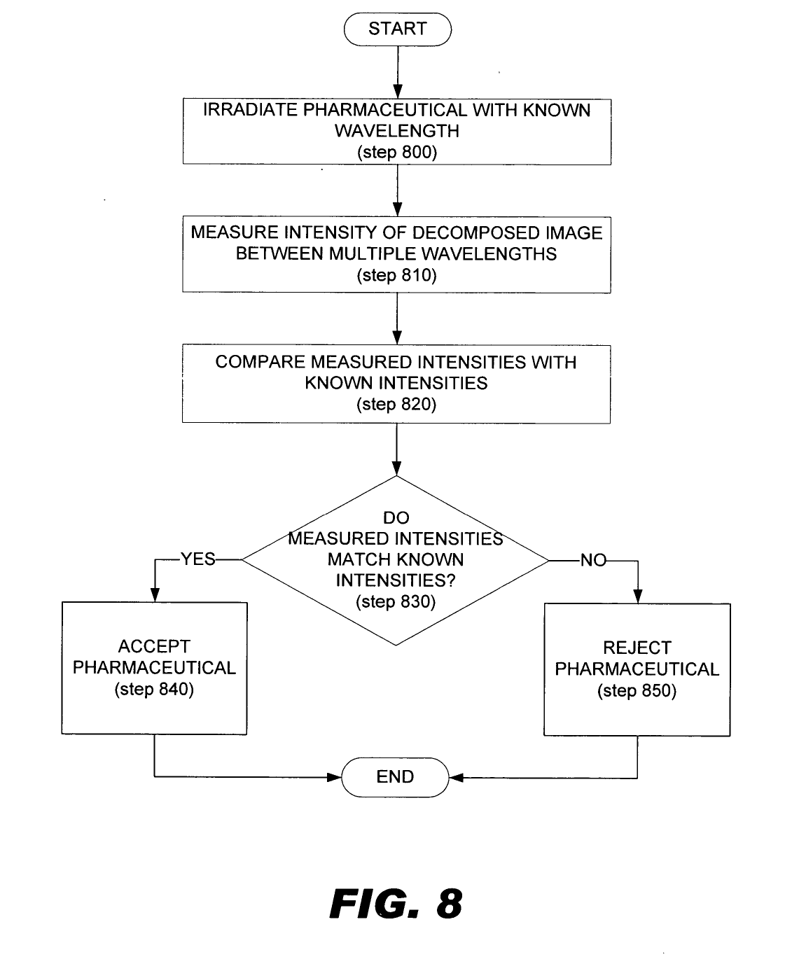

FIG. 8 illustrates an exemplary method for authenticating a pharmaceutical that has been marked with the present marking system, according to one exemplary embodiment. As illustrated in FIG. 8, the present exemplary method begins by first irradiating the pharmaceutical with a known wavelength (step 800). Once the marked pharmaceutical has been irradiated (step 800), the intensity of the decomposed image on the pharmaceutical may be examined and measured (step 810). With the intensity of the decomposed image on the pharmaceutical known at various wavelengths, the measured intensities are compared to known intensities for the identified pharmaceutical (step 820). If the measured intensities substantially match the known intensities (YES, step 830), the pharmaceutical is accepted as genuine (step 840). In contrast, if the measured intensities do not match the known intensities (NO, step 830), the pharmaceutical may be rejected (step 850) as tainted or counterfeit.

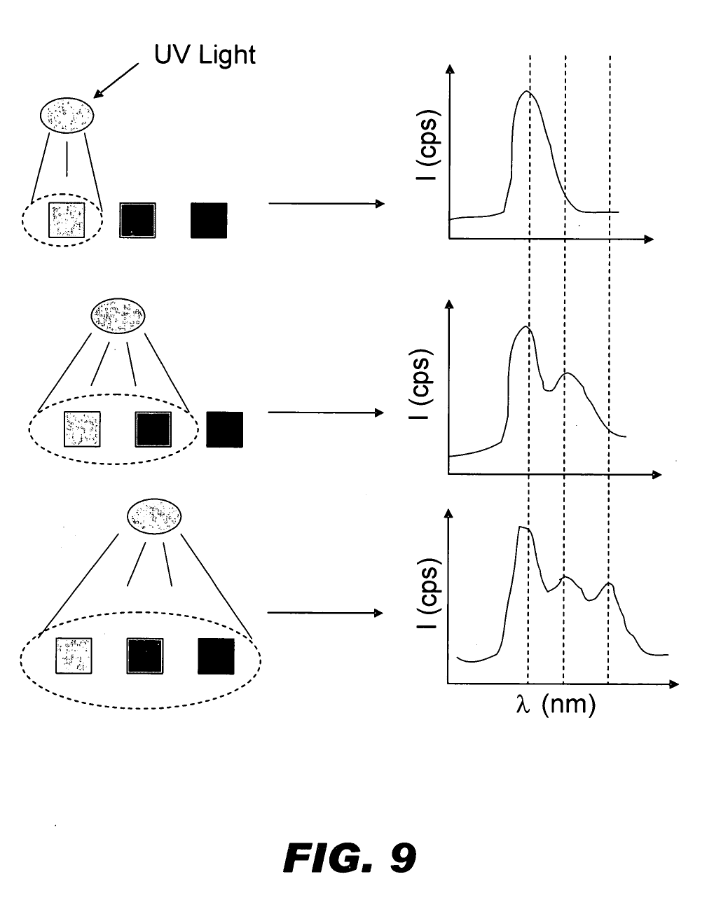

FIG. 9 illustrates another exemplary method for marking a desired substrate. As illustrated in FIG. 9, the various UV fluorescing inks may be spatially distributed on a substrate being marked. According to this exemplary embodiment illustrated in FIG. 9, the resulting spectral fingerprint of the irradiated ink may depend on the optical view of the UV light irradiating the fluorescing ink. As illustrated in FIG. 9, when the optical view of the UV light irradiates a single ink deposition, the resulting spectral fingerprint corresponds to the single ink deposition irradiated. As additional spatially deposited inks are irradiated by the UV light, the resulting spectral fingerprint changes as well, as shown in FIG. 9.

Marking pharmaceutical solid dosage forms with a spectral fingerprint of fluorescent compounds will help track fraudulent dispensing of drugs, protect drugs from counterfeiting, and ensure that patients receive the right medication without affecting the product's appearance.

In an alternative embodiment, the present edible invisible ink may be used to mark a urethane polymer covering or other pharmaceutical packaging of a pharmaceutical. Pharmaceuticals are often packaged in a urethane polymer covering referred to as a blister coating. Similar to the restrictions placed on pharmaceuticals, the FDA restricts the colorants that may be used to mark pharmaceutical packaging. According to one exemplary embodiment, the present system and method is used to mark a urethane polymer covering or other pharmaceutical covering with an edible ink. According to this embodiment, a product name, dosage, barcode, or any other marking may be printed on the urethane polymer coating using the methods explained above. Once printed, the edible ink will not obstruct the view of a contained pharmaceutical or otherwise deface the urethane polymer covering when viewed under normal white light conditions. However, when exposed to UV light, the edible ink fluoresces in the visible light range exposing a spectral fingerprint.

In yet an additional alternative embodiment, the above-mentioned systems and methods may be used to mark and/or track any number of substrates. According to this exemplary embodiment, any number of UV fluorescing inks having distinct fluorescent emission profiles may be used for industrial applications. More specifically, inks having known fluorescent emission profiles may be applied to any substrate including, but not limited to, packaging. According to this exemplary embodiment, the UV fluorescing inks may include non-edible inks for a number of non-oral applications. Consequently, the UV fluorescing inks may be dispensed onto the desired substrate in any number of formations.

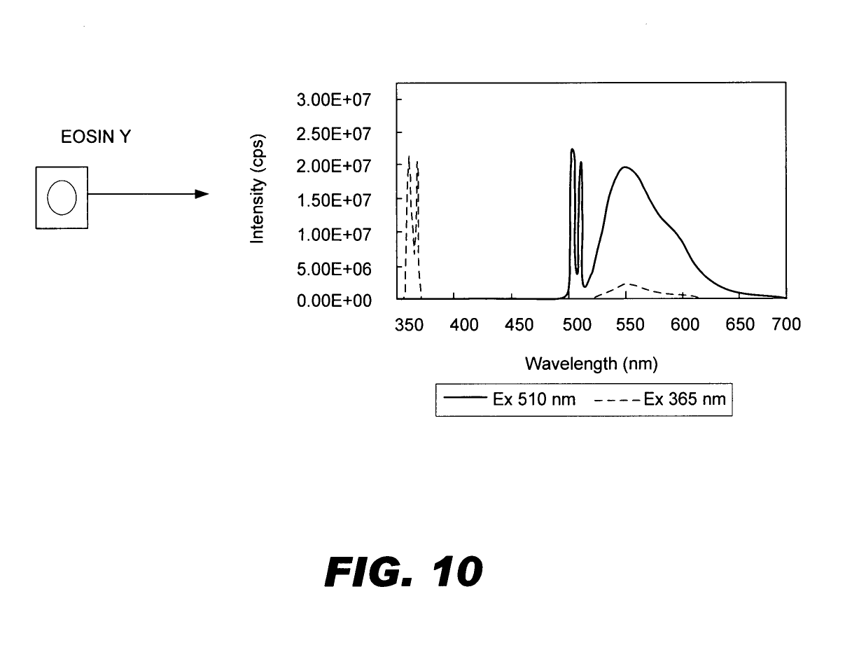

Alternatively, a pharmaceutical may be marked by any number of invisible fluorescent inks that fluoresce in the visible range when exposed to a known range of visible light. For example FIG. 10 illustrates a fluorescence intensity of Eosin Y as a function of excitation wavelength. According to the exemplary embodiment shown in FIG. 10, exposure of Eosin Y to visible light having a wavelength of approximately 510 nm causes the Eosin Y to fluoresce. As illustrated in FIG. 10, the Eosin Y exhibits an intensity of approximately 2.30E+07 cps at its peak when exposed to an excitation wavelength of approximately 510 nm. Specifically, according to this alternative embodiment, exposing the colorant to visible light causes the invisible colorant to fluoresce in the visible region between approximately 500 and 700 nm. By incorporating an ink that fluoresces in the visible region between approximately 500 and 700 nm, authentication may be optically performed when exposed to a known excitation wavelength. As illustrated in FIG. 10, the fluorescence intensity of the Eosin Y when exposed to an excitation wavelength of approximately 510 nm (2.30E+07 cps) is greater than the intensity produced when exposed to an ultraviolet wavelength of approximately 365 nm (2.10E+07 cps). Since authentication may be optically performed at higher intensities when using the invisible fluorescent inks that fluoresce in the visible range when exposed to a known range of visible light, smaller amounts of colorant may be used in the ink formulation compared to the formulations that are analyzed by a spectrofluorometer.

According to the present alternative embodiment, the invisible fluorescent inks configured to fluoresce in the visible range when exposed to a known range of visible light may include, but are in no way limited to, fluorescein-based dyes such as Eosin, Erythrosine, and Phloxine, all of which are edible and appropriate for application to pharmaceutical products. Further, the present fluorescein-based dyes may be combined with the above-mentioned ink vehicles to form an inkjettable ink.

Moreover, the present invisible fluorescent inks configured to fluoresce in the visible range when exposed to a known range of visible light may be combined with one or more of the above-mentioned UV fluorescing inks to form a fluorescent emission profile having a plurality of peaks that may be authenticated by being examined by a spectrofluorometer. According to this exemplary embodiment, the use of an ink that fluoresces in the visible wavelength range may be used as an initial authentication measure, and the one or more UV fluorescing inks may be incorporated as a secondary authentication and/or tracking measure.

In conclusion, the present system and method for implementing an edible ink provide a way to mark pharmaceuticals with “invisible” unique marks to control fraudulent dispensing of counterfeit drugs and to aid in the dispensing and administration of drugs, while reducing medication errors. Such inks are designed for a unique spectral profile when excited with illumination of a known wavelength. The use of multiple inks in combined patterns to authenticate pharmaceutical products is difficult to duplicate. In addition, data can be encoded within these patterns to enable product tracking features. The present edible ink may be safely used to print or otherwise mark on pharmaceutical substrates such as tablets, capsules, gel caps, pills, caplets, and other solid dosage forms; dental products and instruments; and or food products.

Products may be marked by the present edible ink with information such as, but not limited to, logos, names, bar codes, alphanumeric codes, text, watermarks, and other markings. Marking pharmaceuticals with information using invisible ink allows manufacturers and distributors to control fraudulent dispensing of drugs, control counterfeit production of drugs, and ensure that patients receive the correct medication, among other things.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method for labeling a substrate comprising:
positioning said substrate adjacent to an ink material dispenser; and
selectively depositing an ink onto said substrate with said ink material dispenser;
wherein said ink is configured to exhibit a known fluorescent emissive profile when exposed to a visible light.

2. The method of claim 1, wherein said ink comprises:
a jettable vehicle; and
a colorant configured to exhibit a visible fluorescent emissive profile when exposed to said visible light.

3. The method of claim 2, wherein said colorant comprises a plurality of colorants combined in said jettable vehicle, at least one of said plurality of colorants including a colorant configured to exhibit a visible fluorescent emissive profile when exposed to a visible light;
wherein said combined colorants are configured to exhibit a known fluorescent emissive profile when exposed to a known light combination including said visible light.

4. The method of claim 1, wherein;
said ink is edible; and
said substrate comprises one of a pharmaceutical product, a food product, a pharmaceutical covering, or a dental product.

5. The method of claim 1, wherein said visible light comprises a wavelength between approximately 500 and 550 nm.

6. The method of claim 5, wherein said visible light comprises a wavelength of approximately 510 nm.

7. The method of claim 1, wherein said selectively dispensing comprises:
generating a desired image using a computing device;
translating said desired image into a plurality of ink dispenser commands; and
selectively dispensing said ink onto said substrate in a pattern corresponding to said desired image.

8. The method of claim 7, wherein said desired image comprises one of a barcode, a data matrix, a trademark, a trade name, or a dosage indicator.

9. The method of claim 1, wherein said ink is invisible when viewed under white light conditions.

10. The method of claim 1, wherein said ink comprises a fluorescein-based dye.

11. The method of claim 10, wherein said fluorescein-based dye comprises one of Eosin, Erythrosine, or Phloxine.

12. The method of claim 1, wherein said ink is configured to fluoresce at a visible wavelength between approximately 520 and 700 nm when exposed to said visible light.

13. A method for labeling a pharmaceutical product comprising:
positioning said pharmaceutical product adjacent to a inkjet material dispenser; and
selectively jetting an edible ink onto said pharmaceutical product with said inkjet material dispenser;
wherein said edible ink is configured to exhibit a visible fluorescent emissive profile when exposed to a visible light wave.

14. The method of claim 13, wherein said selectively jetting an edible ink onto said pharmaceutical product comprises jetting said edible ink in the form of a desired image.

15. The method of claim 13, wherein said desired image comprises one of a barcode, a data matrix, a trademark, a trade name, or a dosage indicator.

16. The method of claim 13, wherein:
said visible fluorescent emissive profile exhibits a wavelength between approximately 520 and 700 nm; and
said visible light has a wavelength between approximately 500 and 550 nm.

17. A method for marking a pharmaceutical product, comprising:
positioning said pharmaceutical product adjacent to a first inkjet material dispenser;
selectively jetting a first edible ink onto said pharmaceutical product with said first inkjet material dispenser;
positioning said pharmaceutical product adjacent to a second inkjet material dispenser; and
selectively jetting a second edible ink onto said first edible ink with said second inkjet material dispenser;
wherein said first edible ink is configured to exhibit a visible emissive profile when exposed to a visible light; and
said second edible ink is configured to exhibit a known fluorescent emissive profile when exposed to an ultraviolet light.

18. The method of claim 17, wherein said pharmaceutical product comprises one of a tablet, a capsule, a gel cap, a caplet, a pill, or a pharmaceutical covering.

19. The method of claim 17, wherein said first inkjet material dispenser and said second inkjet material dispenser each comprise one of a thermally actuated inkjet dispenser, a mechanically actuated inkjet dispenser, an electrostatically actuated inkjet dispenser, a magnetically actuated dispenser, a piezoelectrically actuated dispenser, or a continuous inkjet dispenser.

20. The method of claim 17, wherein said selectively jetting a first edible ink onto said pharmaceutical product with said first inkjet material dispenser further comprises printing one of a barcode, a data matrix, a trademark, a trade name, or a dosage indicator.

21. A method for tracking a fraudulent dispensing of pharmaceuticals, comprising:
marking a pharmaceutical with an edible ink, wherein said edible ink is configured to exhibit a known visible emissive profile when exposed to a visible light;
exposing said pharmaceutical to said visible light; and
analyzing a fluorescent emissive profile of said edible ink to authenticate said pharmaceutical.

22. The method of claim 21, wherein said step of analyzing said fluorescent emissive profile comprises examining said fluorescent emissive profile with a spectrofluorometer.

23. The method of claim 22, wherein said step of analyzing said fluorescent emissive profile comprises:
acquiring a fluorescent emissive profile of said edible ink; and
comparing said emissive profile to a known emissive profile.

24. A method for controlling an administration of proper medication to patients, comprising:
marking a pharmaceutical with information in an edible ink, wherein said edible ink is configured to exhibit a known fluorescent emissive profile when exposed to a visible light having a wavelength between approximately 500 and 550 nm; and
exposing said pharmaceutical to said visible light to verify said information.

25. The method of claim 24, wherein said step of exposing said pharmaceutical to said visible light to verify said information further comprises:
acquiring a fluorescent emissive profile of said edible ink; and
comparing said emissive profile to a known emissive profile.

26. A pharmaceutical comprising:
a surface;
wherein said surface is marked with an edible ink;
said edible ink being configured to exhibit a known fluorescent emissive profile when exposed to a visible light.

27. The pharmaceutical of claim 26, wherein said mark comprises one of a barcode, a data matrix, a trademark, a trade name, or a dosage indicator.

28. The pharmaceutical of claim 26, wherein said edible ink comprises a fluorescein-based dye.

29. A system for labeling a pharmaceutical comprising:
a pharmaceutical carrying substrate;
an inkjet material dispenser disposed adjacent to said pharmaceutical carrying substrate;
an ink reservoir associated with said inkjet material dispenser wherein said ink reservoir is configured to supply an edible ink to said inkjet material dispenser, said edible ink being configured to exhibit a known fluorescent emissive profile when exposed to a visible light; and
a vision system disposed adjacent to said pharmaceutical carrying substrate, said vision system being configured to analyze an emissive profile of said edible ink.

30. The system of claim 29, further comprising:
a computing device controllably coupled to said system;
wherein said computing device is configured to control a selective dispensing of said edible ink by said inkjet material dispenser.

31. The system of claim 30, wherein said computing device is further configured to:
receive an emissive profile acquired by said vision system; and
compare said received emissive profile to a known emissive profile.

32. The system of claim 30, wherein said vision system comprises a spectrofluormeter.

33. A system for labeling a pharmaceutical comprising:
means for transporting a pharmaceutical;
means for dispensing an ink, said means for dispensing an ink being disposed adjacent to said means for transporting a pharmaceutical;
means for housing said ink associated with said means for dispensing an ink, wherein said means for housing said ink is configured to supply an edible ink to said means for dispensing an ink, said edible ink being configured to exhibit a known fluorescent emissive profile when exposed to a visible light having a wavelength between approximately 500 and 550 nm; and
means for examining an emissive profile of said edible ink, said means for examining being disposed adjacent to said means for transporting a pharmaceutical.

34. The system of claim 33, further comprising:
means for computing data controllably coupled to said system;
wherein said computing device is configured to control a selective dispensing of said edible ink by said ink dispensing means.

35. The system of claim 34, wherein said computing means is further configured to:
receive an emissive profile acquired by said examining means; and
compare said received emissive profile to a known emissive profile.

36. The system of claim 33, wherein said examining means comprises a spectrofluormeter.
